(19) United States
(12) Patent Application Publication
(10) Pub. No.: US 2026/0240661 A1
(43) Pub. Date: Aug. 20, 2026

(54) MINIMALLY INVASIVE SPINAL FUSION IMPLANT SYSTEMS AND METHODS

(71) Applicant: Sharp Biomedical Corp., Edina, MN (US)

(72) Inventors: Dan NEWMAN, Edina, MN (US); James Jeffrey ROBBINS, Logan, UT (US); Jonathan CLARK, JR., Hyde Park, UT (US); Nathan William ERICKSON, Beaverdam, UT (US)

(73) Assignee: Sharp Biomedical Corp., Edina, MN (US)

(21) Appl. No.: 19/542,596

(22) Filed: Feb. 17, 2026

Related U.S. Application Data
(60) Provisional application No. 63/759,185, filed on Feb. 16, 2025.

(51) Int. Cl. A61B 17/70

(52) U.S. Cl. CPC A61F 2/4405; A61B 17/7062; A61B 17/7065; A61B 17/7068; A61F 2/30749; A61F 2/30771; A61F 2/446; A61F 2002/3008; A61F 2002/30518; A61F 2002/30576; A61F 2002/30601; A61F 2002/3093; A61F 2002/4687

(57) ABSTRACT

A system for stabilizing a joint between a superior vertebra and an inferior vertebra may include an implant having a body configured to be implanted between a superior spinous process of the superior vertebra and an inferior spinous process of the inferior vertebra, and a facet arm having a facet fixation feature, wherein the facet arm may be movable between a retracted configuration in which the facet arm may be housed within the body, and a deployed configuration in which the facet arm may extend laterally from the body to position the facet fixation feature in alignment with a facet joint to facilitate coupling of the facet arm to the facet joint.

### CROSS-REFERENCE TO RELATED APPLICATIONS

The present application claims the benefit of U.S. Provisional Patent Application Ser. No. 63/759,185, filed on Feb. 16, 2025 and entitled SPINAL IMPLANT FOR SECURING FACET JOINTS AND FACILITATING MULTI-AXIS INTERSPINOUS PROCESS FUSION, which is incorporated by reference as though set forth herein in its entirety.

### TECHNICAL FIELD

The present disclosure relates generally to surgical systems and methods, and more specifically, to systems and methods for maintaining a desired level of distraction between spinous processes and laminae of adjacent vertebrae.

### BACKGROUND

Spinal fusion procedures are commonly performed to stabilize spinal segments and reduce pain associated with conditions such as degenerative disc disease, spinal stenosis, spondylolisthesis, and other forms of spinal instability. Conventional spinal fusion techniques typically require open or highly invasive surgical approaches that involve extensive tissue disruption, retraction of musculature, and removal of bone. Such approaches may increase operative trauma, prolong recovery time, and elevate the risk of post-operative complications.

In response, minimally invasive spinal fusion techniques have been developed to reduce surgical morbidity and preserve surrounding anatomy. However, many existing minimally invasive systems provide limited stabilization, rely on complex or bulky instrumentation, or fail to adequately address both decompression and fixation of the affected spinal segment. In particular, some systems do not effectively secure the facet joints or provide multi-axis stabilization while maintaining a minimally invasive deployment profile. Accordingly, there remains a need for improved spinal fusion implants and methods that enable effective decompression, stabilization, and fusion of the spine through a minimally invasive approach.

### SUMMARY

The various systems and methods of the present disclosure have been developed in response to the present state of the art, and in particular, in response to the problems and needs in the art that have not yet been fully solved by currently available minimally invasive spinal fusion implant systems and methods.

In some embodiments, a system for stabilizing a joint between a superior vertebra and an inferior vertebra may include an implant having a body configured to be implanted between a superior spinous process of the superior vertebra and an inferior spinous process of the inferior vertebra, and a facet arm having a facet fixation feature, wherein the facet arm may be movable between a retracted configuration in which the facet arm may be housed within the body, and a deployed configuration in which the facet arm may extend laterally from the body to position the facet fixation feature in alignment with a facet joint to facilitate coupling of the facet arm to the facet joint.

In the system of any preceding paragraph, in the retracted configuration, the implant may be configured to pass through a cannula to engage the superior spinous process and the inferior spinous process.

In the system of any preceding paragraph, in the deployed configuration, the implant may be sized to inhibit passage through the cannula.

In the system of any preceding paragraph, the body may include an internal cavity configured to receive bone graft material.

In the system of any preceding paragraph, the system may further include a fastener configured to be received in the facet joint, wherein the facet fixation feature may include an aperture configured to receive the fastener, and in the deployed configuration, the fastener may be insertable through the aperture and into the facet joint.

In the system of any preceding paragraph, the implant may further include a wing mechanism configured to engage the superior spinous process and the inferior spinous process.

In the system of any preceding paragraph, the system may further include a guided access device configured to be docked with an interspinous space between the superior vertebra and the inferior vertebra, wherein the guided access device may include a cannula configured to receive the implant and direct the implant to the interspinous space.

In the system of any preceding paragraph, the facet fixation feature may include an aperture configured to be aligned with the facet joint, and the guided access device may further include a trajectory guide comprising a longitudinal axis that may be aligned with the aperture.

In some embodiments, a system for stabilizing a joint between a superior vertebra and an inferior vertebra may include an implant having a body, a wing mechanism configured to engage a superior spinous process and an inferior spinous process, and a first actuation mechanism configured to engage the wing mechanism, wherein actuation of the first actuation mechanism may expand at least a portion of the body to distract an interspinous space between the superior vertebra and the inferior vertebra, and further actuation of the first actuation mechanism may deploy the wing mechanism to engage the superior spinous process and the inferior spinous process.

In the system of any preceding paragraph, the implant may further include a facet arm having a facet fixation feature configured to facilitate coupling of the facet arm to a facet joint.

In the system of any preceding paragraph, the implant may further include a second actuation mechanism configured to engage the facet arm, wherein actuation of the second actuation mechanism may extend the facet arm laterally relative to the body to position the facet fixation feature in alignment with a facet joint.

In the system of any preceding paragraph, the facet arm may be movable between a retracted configuration in which the facet arm may be housed within the body, and a deployed configuration in which the facet arm may extend laterally from the body to position the facet fixation feature in alignment with a facet joint to facilitate coupling of the facet arm to the facet joint.

In the system of any preceding paragraph, the system may further include a fastener configured to be received in the facet joint, wherein the facet fixation feature may include an aperture configured to receive the fastener, and in the deployed configuration, the fastener may be insertable through the aperture and into the facet joint.

In the system of any preceding paragraph, the system may further include a guided access device configured to be docked with the interspinous space, wherein the guided access device may include a cannula configured to receive the implant and direct the implant to the interspinous space.

In the system of any preceding paragraph, the implant may further include a facet arm having a facet fixation feature configured to facilitate coupling of the facet arm to a facet joint, the facet fixation feature may include an aperture configured to be aligned with the facet joint, and the guided access device may further include a trajectory guide having a longitudinal axis that may be aligned with the aperture.

In some embodiments, a system for stabilizing a joint between a superior vertebra and an inferior vertebra may include an implant having a body configured to be implanted between a superior spinous process of the superior vertebra and an inferior spinous process of the inferior vertebra, and a first facet arm, a second facet arm, and a wing mechanism configured to engage the superior spinous process and the inferior spinous process. The system may further include a guided access device configured to be docked with an interspinous space between the superior vertebra and the inferior vertebra. The guided access device may include a cannula configured to receive the implant and direct the implant to the interspinous space, the implant may be configured to pass through the cannula to engage the superior vertebra and the inferior vertebra, the first facet arm may include a first aperture configured to be aligned with a first facet joint, the second facet arm may include a second aperture configured to be aligned with a second facet joint, the guided access device may further includes a first trajectory guide comprising a first longitudinal axis that may be alignable with the first aperture, and the guided access device may further include a second trajectory guide having a second longitudinal axis that is alignable with the second aperture.

In the system of any preceding paragraph, the system may further include a first fastener configured to be received in the first facet joint, and a second fastener configured to be received in the second facet joint, wherein the first fastener may be insertable through the first aperture and into the first facet joint, and the second fastener may be insertable through the second aperture and into the second facet joint.

In the system of any preceding paragraph, the implant may further include a first actuation mechanism configured to engage the wing mechanism, wherein actuation of the first actuation mechanism may expand at least a portion of the body to distract the interspinous space between the superior vertebra and the inferior vertebra, and further actuation of the first actuation mechanism may deploy the wing mechanism to engage the superior spinous process and the inferior spinous process.

In the system of any preceding paragraph, the implant may further include a second actuation mechanism configured to engage the first facet arm and the second facet arm, wherein actuation of the second actuation mechanism may extend the first facet arm laterally in a first direction relative to the body to position the first aperture in alignment with the first facet joint, and actuation of the second actuation mechanism may extend the second facet arm laterally in a second direction opposite the first direction relative to the body to position the second aperture in alignment with the second facet joint.

In the system of any preceding paragraph, the first facet arm may be movable between a retracted configuration in which the first facet arm may be housed within the body, and a deployed configuration in which the first facet arm may extend laterally in a first direction from the body to position the first aperture in alignment with the first facet joint to facilitate coupling of the first facet arm to the first facet joint. The second facet arm may be movable between the retracted configuration in which the second facet arm may be housed within the body, and the deployed configuration in which the second facet arm may extend laterally in a second direction opposite the first direction from the body to position the second aperture in alignment with the second facet joint to facilitate coupling of the second facet arm to the second facet joint.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings.

FIG. 1A is a perspective view of a minimally invasive spinal fusion implant according to an embodiment of the present disclosure.

FIG. 1B is a perspective exploded view of the minimally invasive spinal fusion implant of FIG. 1A.

FIG. 1C is a perspective view an implant body and wings of the minimally invasive spinal fusion implant of FIG. 1A according to an embodiment of the present disclosure.

FIG. 2A is a perspective view of the implant body of FIG. 1C according to an embodiment of the present disclosure.

FIG. 2B is a section view of the implant body of FIG. 2A.

FIG. 2C is a section view of the implant body of FIG. 2A.

FIG. 3A is a top view of the minimally invasive spinal fusion implant of FIG. 1A in a retracted configuration according to an embodiment of the present disclosure.

FIG. 3B is a top view of the minimally invasive spinal fusion implant of FIG. 1A in a partially deployed configuration according to an embodiment of the present disclosure.

FIG. 4 is a top view of the minimally invasive spinal fusion implant of FIG. 1A in a deployed configuration according to an embodiment of the present disclosure.

FIG. 5A is a perspective view of a minimally invasive spinal fusion implant according to an embodiment of the present disclosure.

FIG. 5B is a perspective exploded view of the minimally invasive spinal fusion implant of FIG. 5A.

FIG. 5C is a perspective view an implant body and wings of the minimally invasive spinal fusion implant of FIG. 5A according to an embodiment of the present disclosure.

FIG. 6A is a perspective view of a first wing of the minimally invasive spinal fusion implant of FIG. 5A according to an embodiment of the present disclosure.

FIG. 6B is a perspective view of the first wing of FIG. 6A.

FIG. 7A is a perspective view of the implant body of FIG. 5C according to an embodiment of the present disclosure.

FIG. 7B is a section view of the implant body of FIG. 7A.

FIG. 7C is a section view of the implant body of FIG. 7A.

FIG. 8 is a perspective view of a guided access device having a cannula, docked with an interspinous space between a superior vertebra and an inferior vertebra according to an embodiment of the present disclosure.

FIG. 9 is a perspective view of a driver received in the guided access device of FIG. 8 according to an embodiment of the present disclosure.

FIG. 10 is a perspective view of the guided access device of FIG. 8 having a trajectory guide and guide pins according to an embodiment of the present disclosure.

FIG. 11 is a perspective view of the guided access device of FIG. 10 and bone fasteners according to an embodiment of the present disclosure.

FIG. 12A is a perspective view of an implant body of a minimally invasive spinal fusion implant according to an embodiment of the present disclosure.

FIG. 12B is a side view of the implant body of FIG. 12A.

FIG. 13A is a perspective view of an actuation mechanism, an anchor block, and a retention wing of the implant body of FIG. 12A according to an embodiment of the present disclosure.

FIG. 13B is a side view of the actuation mechanism, the anchor block, and the retention wing of FIG. 13A.

FIG. 13C is a side view of the actuation mechanism of FIG. 13A.

FIG. 13D is a perspective view of the anchor block and the retention wing of FIG. 13A.

FIG. 14A is a perspective view of a minimally invasive spinal fusion implant in a retracted configuration according to an embodiment of the present disclosure.

FIG. 14B is a perspective view of the minimally invasive spinal fusion implant of FIG. 14A in a deployed configuration according to an embodiment of the present disclosure.

FIG. 15A is a top section view of the minimally invasive spinal fusion implant of FIG. 14B in a deployed configuration.

FIG. 15B is a top section view of the minimally invasive spinal fusion implant of FIG. 14B in a deployed configuration.

FIG. 16 is a perspective view of the minimally invasive spinal fusion implant of FIG. 14A deployed in an interspinous space between a superior vertebra and an inferior vertebra according to an embodiment of the present disclosure.

FIG. 17 is a perspective view of the minimally invasive spinal fusion implant of FIG. 14A deployed in an interspinous space between a superior vertebra and an inferior vertebra according to an embodiment of the present disclosure.

FIG. 18A is a perspective view of a minimally invasive spinal fusion implant and bone fasteners according to an embodiment of the present disclosure.

FIG. 18B is a perspective view of a first clamp member of the minimally invasive spinal fusion implant of FIG. 18A according to an embodiment of the present disclosure.

FIG. 18C is a perspective view of a second clamp member of the minimally invasive spinal fusion implant of FIG. 18A according to an embodiment of the present disclosure.

FIG. 19A is a perspective view of the bone fastener of FIG. 18A.

FIG. 19B is a perspective view of a minimally invasive spinal fusion implant and bone fasteners according to an embodiment of the present disclosure.

FIG. 20 is a perspective view of the minimally invasive spinal fusion implant and bone fasteners of FIG. 19B deployed in an interspinous space between a L5 vertebra and a sacrum according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to spinal fusion implant systems. Those skilled in the art will recognize that the following description is merely illustrative of the principles of the technology, which may be applied in various ways to provide many alternative embodiments. For the purposes of illustrating the concepts of the present design, the present disclosure describes a minimally invasive spinal fusion implant system including expandable wings to facilitate securing of facet joints. However, it will be understood that other variations and uses are contemplated, including, but not limited to, other means of securing facet joints and/or securing an implant to adjacent spinous processes.

Referring to the figures, various aspects of the minimally invasive interspinous fusion system will be described in detail. It will be understood that the described embodiments are exemplary, and modifications and substitutions can be made within the scope of the invention.

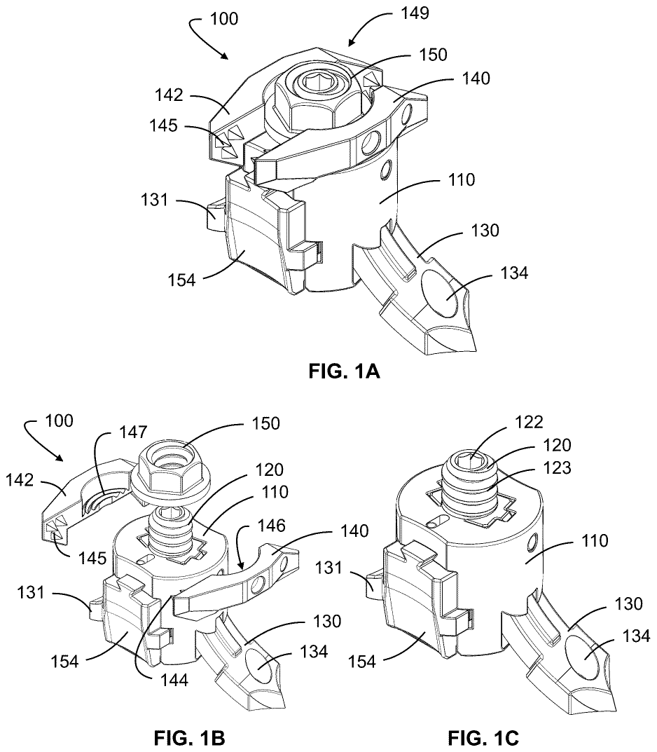

FIG. 1A is a perspective view of a minimally invasive spinal fusion implant 100 according to an embodiment of the present disclosure. FIG. 1B is a perspective exploded view of the minimally invasive spinal fusion implant 100. FIG. 1C is a perspective view an implant body 110, a first facet arm 130, and a second facet arm 131 of the minimally invasive spinal fusion implant 100 according to an embodiment of the present disclosure.

The minimally invasive spinal fusion implant 100 may include an implant body 110, an actuation mechanism 120, a first facet arm 130, a second facet arm 131, a first wing 140, a second wing 142, a locking component 150, and a first distraction portion 154. The first wing 140 and the second wing 142 may cooperate to function as a wing mechanism 149 configured to engage the superior spinous process and the inferior spinous process.

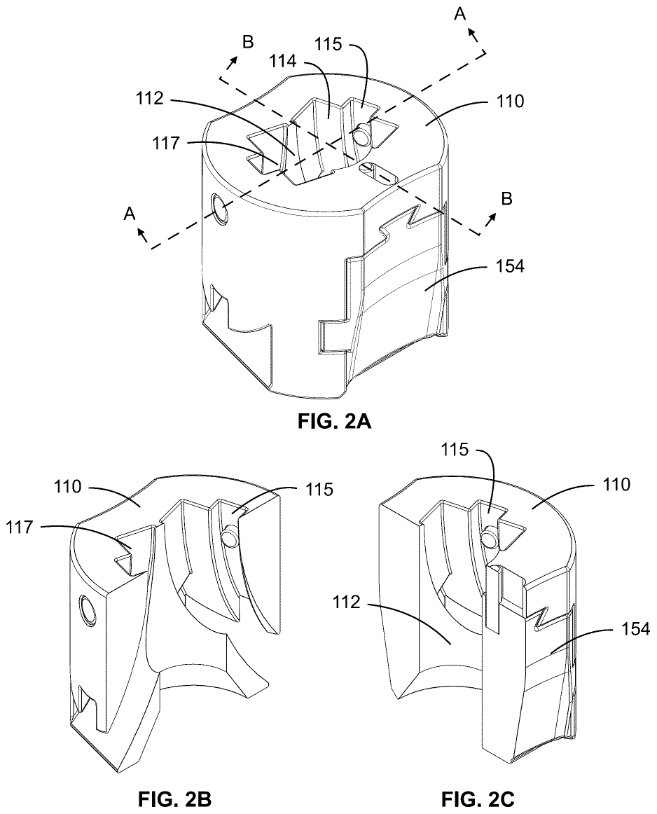

FIG. 2A is a perspective view of the implant body 110 according to an embodiment of the present disclosure. FIG. 2B is a section view of the implant body 110. FIG. 2C is a section view of the implant body 110.

The implant body 110 may include an internal cavity 112, a posterior opening 114, a first facet arm channel 115, and a second facet arm channel 117. The actuation mechanism 120 may include a drive feature 122 and a threaded portion 123. The first facet arm 130 and the second facet arm 131 may be housed within the implant body 110.

The first facet arm 130 may include a first facet arm proximal portion 132, a first facet fixation feature 134, a first facet arm distal portion 136, and a first facet arm protrusion 138. Similarly, the second facet arm 131 may include a second facet arm proximal portion 133, a second facet fixation feature 135, a second facet arm distal portion 137, and a second facet arm protrusion 139. The facet arms 130, 131 may be movably coupled to the implant body 110 within the facet arm channels 115, 117 and may be configured to move between a retracted configuration and a deployed configuration.

The first wing 140 may include a plurality of first wing bone engaging features 144 and a first wing engagement portion 146. The second wing 142 may include a plurality of second wing bone engaging features 145 and a second wing engagement portion 147.

With reference to FIGS. 1A, 1B, 1C, the implant 100 may include the implant body 110 configured to reside between two adjacent vertebral spinous processes. More specifically, the implant 100 may be configured to be implanted between a superior spinous process of a superior vertebra 10 and an inferior spinous process of an inferior vertebra 20. The implant body 110 may be a solid monolithic member and/or may define an internal cavity 112 (FIGS. 2B-2C) for bone graft.

In one embodiment, the interior of the implant body 110 may form an internal cavity 112 open to the front. The internal cavity 112 may be a hollow interior cavity configured to receive bone graft material such as demineralized bone matrix, allograft, autograft, or other osteoconductive or osteoinductive materials. The posterior opening 114 may provide access to the internal cavity 112 for packing bone graft material. The first facet arm channel 115 and second facet arm channel 117 may be configured to receive and guide the first facet arm 130 and second facet arm 131, respectively.

The implant body 110 may include radiopaque markers to facilitate imaging during surgery. In some designs, portions of the body may be additively manufactured with a porous or lattice structure to promote bone ingrowth. The body may further include protruding flange or prong features to enhance mechanical interlock with bone.

The implant body 110 may be formed from biocompatible materials including, but not limited to, titanium alloys (such as Ti—6Al—4V), stainless steel, medical-grade polymers (such as PEEK or PEKK), carbon-fiber-reinforced polymers, or composite materials. The body dimensions and profile may be tailored to fit within the interspinous space. The inferior surface of the implant body may be typically shaped to seat against the upper border of the inferior spinous process, and the superior surface to engage the lower border of the superior spinous process when implanted.

The actuation mechanism 120 may extend axially through the implant body 110 and may include the drive feature 122 and the threaded portion 123. The drive feature 122 may be positioned at a proximal end of the actuation mechanism 120 and may comprise a hex recess, square socket, Torx recess, or other suitable tool interface configured to engage a surgical driver instrument. The threaded portion 123 may be configured to engage internal threads within the implant body 110 or other components of the implant 100. Actuation of the actuation mechanism 120 may cause expansion and/or distraction of the implant 100, and/or deployment of the first facet arm 130 and/or the second facet arm 131. Further actuation of the actuation mechanism 120 may deploy the first wing 140 and/or the second wing 142 to engage the superior spinous process and the inferior spinous process.

Actuation of the actuation mechanism 120 may cause: (i) telescopic expansion or jack-screw raising of the implant (distracting the spinous processes). Further actuation of the actuation mechanism 120 may further cause: (ii) actuation of linkages that deploy the lamina-engaging wing(s); and/or (iii) advancement of the locking component 150 to tighten the first wing 140 and/or the second wing 142. In some embodiments, the actuation mechanism 120 may be cannulated to allow insertion over a guidewire for percutaneous placement. The actuation mechanism 120 may also include a shearable section or torque-limiting mechanism in embodiments where controlled distraction may be required.

For example, rotating the actuation mechanism 120 clockwise may drive an internal wedge or cam to actuate a first distraction portion 154, and may simultaneously or subsequently engage a cam follower linkage that pushes the first facet arm 130 and/or the second facet arm 131 outward relative to the implant body 110. After the first facet arm 130 and/or the second facet arm 131 are deployed, further rotation of the shaft may then draw the locking component 150 toward the implant body 110, and draw the first wing 140 and the second wing 142 together. The sequence of operations (distract, deploy arms, clamp wings) may be controlled by designing the threads and linkages with appropriate pitch and mechanical advantage.

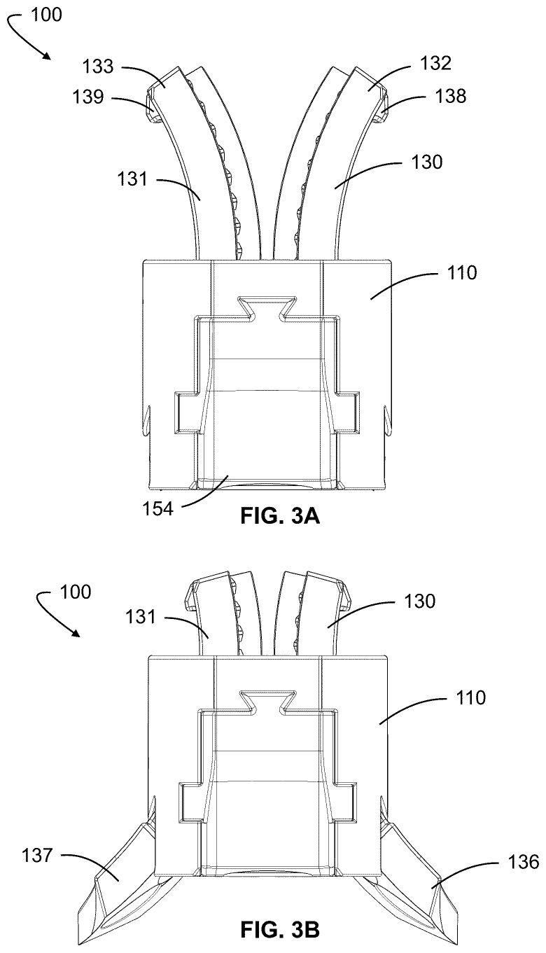

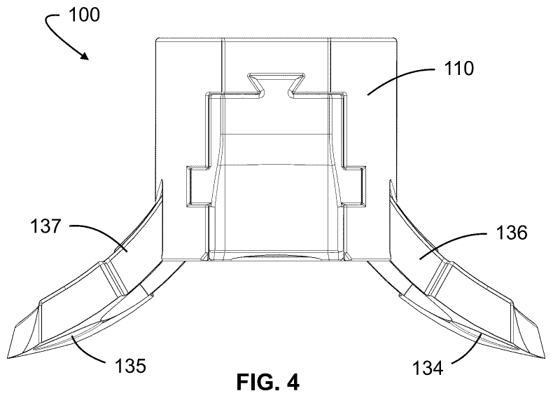

FIG. 3A is a top view of the minimally invasive spinal fusion implant 100 in a retracted configuration according to an embodiment of the present disclosure. FIG. 3B is a top view of the minimally invasive spinal fusion implant 100 in a partially deployed configuration according to an embodiment of the present disclosure. FIG. 4 is a top view of the minimally invasive spinal fusion implant 100 in a deployed configuration according to an embodiment of the present disclosure.

As shown in FIGS. 3A, 3B, and 4, in the retracted configuration (FIG. 3A), the first facet arm 130 and second facet arm 131 may be positioned substantially within or alongside the implant body 110, allowing the implant 100 to pass through a minimally invasive cannula. In the partially deployed configuration (FIG. 3B), the first facet arm 130 and/or the second facet arm 131 may begin to extend laterally outward from the implant body 110. In the fully deployed configuration (FIG. 4), the first facet arm 130 and/or the second facet arm 131 may extend laterally to position the first facet fixation feature 134 and second facet fixation feature 135 in alignment with a first facet joint 50 and a second facet joint 55, respectively, of adjacent vertebrae.

Each of the first facet arm 130 and the second facet arm 131 may have a bone-contacting curved surface sized to engage the inferior aspect of the lamina above. The contacting surface may be roughened or include serrations/teeth to grip bone. In one variant, the wing's underside may be serrated for anti-slip engagement. The wings may be tapered and possibly pointed at the tip to facilitate insertion and/or distraction of soft tissue.

The first facet fixation feature 134 and second facet fixation feature 135 may be configured to receive bone fasteners such as screws or other fixation elements to secure the first facet arm 130 and the second facet arm 131 to the first facet joint 50 and the second facet joint 55, respectively, thereby providing supplemental fixation and stabilization. The first facet arm protrusion 138 and second facet arm protrusion 139 may provide mechanical engagement features and/or mechanical limits that interact with actuation mechanisms to deploy the wings.

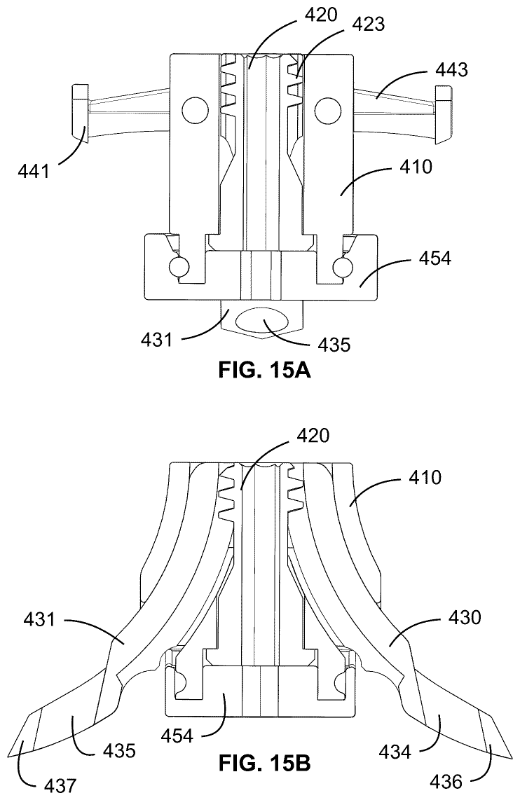

Deployment of the first facet arm 130 and/or the second facet arm 131 may provide secondary fixation. With the first facet arm 130 and/or the second facet arm 131 in the deployed position, relative motion of the vertebrae may be resisted by securing the first facet joint 50 and/or the second facet joint 55. This may counteract flexion, extension, and rotational forces that might otherwise loosen the construct. The first facet arm 130 and/or the second facet arm 131 may be driven by the actuation mechanism 120 through a rack-and-pinion, cam ramp, or lever linkage (FIGS. 3B, 15A-15B). For instance, the actuation mechanism 120 may carry a cam follower that rides along a cam surface on each wing, forcing the wings downward and outward as the actuation mechanism 120 turns. Alternatively, a gear train or slider mechanism driven by the actuation mechanism 120 could push the wings downward and outward.

In alternative embodiments, the first facet arm 130 and/or the second facet arm 131 may be made spring-loaded or powered by a ratchet mechanism. Multiple wings per side (stacked vertically or longitudinally) may also be contemplated. The first facet arm 130 and/or the second facet arm 131 may be formed of the same biocompatible materials as the body.

FIGS. 1A-1C may depict a modular wing mechanism 149 including the first wing 140, the second wing 142, and a locking component 150. Each of the first wing 140 and the second wing 142 may have a body portion that mates with the implant (e.g., slots or pegs that engage the implant) and a first wing bone engaging feature 144 and a second wing bone engaging feature 145 that contact at least one of the superior spinous process and the inferior spinous process. The first wing bone engaging feature 144 and the second wing bone engaging feature 145 may include serrations, teeth, spikes, or other surface features configured to grip and engage the spinous processes.

The first wing 140 and the second wing 142 may be mirror images of each other. The wing engagement portions 146, 147 may be configured to mate with corresponding features on the implant body 110 and may be drawn together by the locking component 150 to clamp the spinous processes securely.

To deploy, the first wing 140 may be placed on one side of the spinous process and the second wing 142 on the opposite side. The locking component 150 may be then tightened onto the actuation mechanism 120. As locking component 150 advances, it may simultaneously push the first wing 140 and the second wing 142 toward each other. The bottom of each wing may ride in a channel or slot of the implant body 110. When fully tightened, the first wing bone engaging feature 144 and the second wing bone engaging feature 145 may bite into the opposing sides of the spinous processes, rigidly clamping the spinous processes against the implant 100. This may lock the implant 100 in place and prevent migration. The locking component 150 may include an anti-backout feature such as a nylon insert, detent mechanism, or other features to maintain the clamping force and prevent loosening.

Because the wing mechanism 149 may be modular, the first wings 140 and the second wing 142 can be detached or reoriented. For example, alternative wing mechanism geometries could include plate-like wings that clamp from above and below the spinous process, or hinged wings. In some embodiments, the wings may be used in a superior/inferior orientation rather than lateral. The wing mechanism 149 can thus be customized to patient anatomy.

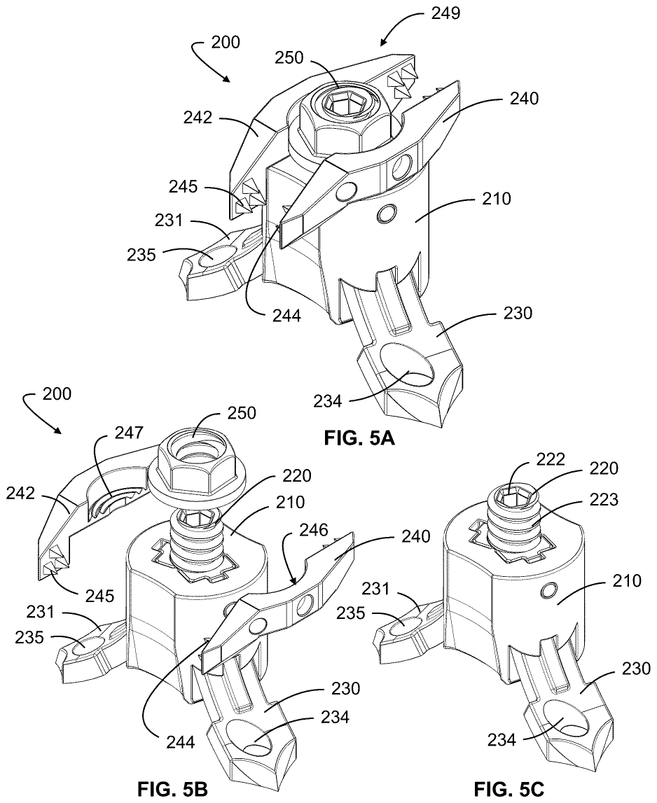

FIG. 5A is a perspective view of a minimally invasive spinal fusion implant 200 according to an embodiment of the present disclosure. FIG. 5B is a perspective exploded view of the minimally invasive spinal fusion implant 200. FIG. 5C is a perspective view an implant body 210 a first facet arm 230, and a second facet arm 231 of the minimally invasive spinal fusion implant 200 according to an embodiment of the present disclosure.

The minimally invasive spinal fusion implant 200 according to a second embodiment may include an implant body 210, an actuation mechanism 220, a first facet arm 230, a second facet arm 231, a first wing 240, a second wing 242, and a locking component 250. The first facet arm 230 may include a first facet fixation feature 234. The second facet arm 231 may include a second facet fixation feature 235. The first wing 240 and the second wing 242 may cooperate to function as a wing mechanism 249 configured to engage the superior spinous process and the inferior spinous process.

The various components of the implant 200 with the same reference numbers as those of the implant 100 may be similar in function and/or configuration to their counterparts of the implant 100. Differences between the implant 200 and the implant 100 will be set forth below.

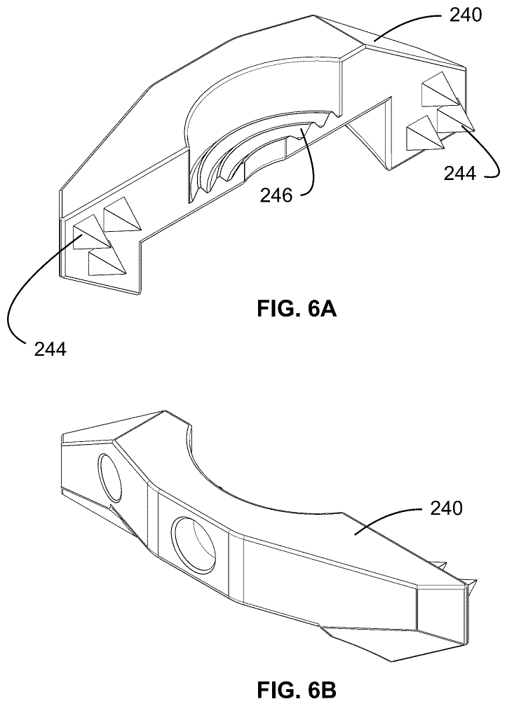

FIG. 6A is a perspective view of a first wing 240 of the minimally invasive spinal fusion implant 200 according to an embodiment of the present disclosure. FIG. 6B is a perspective view of the first wing 240.

The first wing 240 may include a plurality of first wing bone engaging features 244, and a first wing engagement portion 246. The first wing bone engaging features 244 may be configured similarly to the bone engaging features 144 of the first embodiment, with serrations or other surface texturing to grip the spinous process. The first wing engagement portion 246 may be configured to mate with the implant body 210. The first wing engagement portion 246 may provide clearance for the actuation mechanism 220 and/or may serve as an attachment point for the locking component 250.

The second wing 242 may be substantially a mirror image of the first wing 240, facilitating symmetric clamping of the spinous processes from opposing lateral sides. The second wing 242 may include a plurality of second wing bone engaging features 245, and a second wing engagement portion 247. In some embodiments, the wings 240, 242 may be concave or curved to partially wrap around the spinous processes, providing enhanced contact area and clamping force distribution.

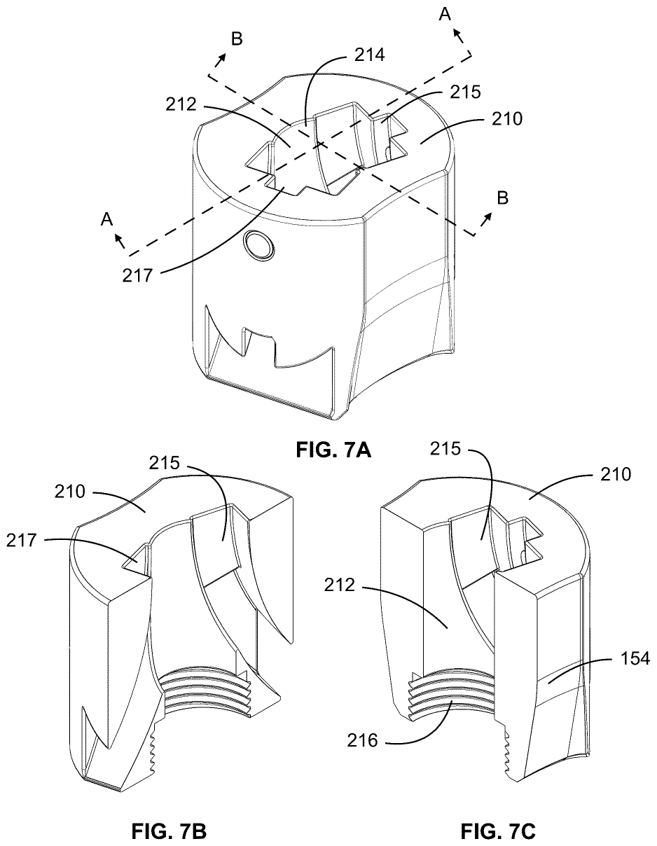

FIG. 7A is a perspective view of the implant body 210 according to an embodiment of the present disclosure. FIG. 7B is a section view of the implant body 210. FIG. 7C is a section view of the implant body 210.

As shown in FIGS. 7A, 7B, and 7C, the implant body 210 may include an internal cavity 212, a posterior opening 214, a first facet arm channel 215, a second facet arm channel 217, and an anterior opening 216. The internal cavity 212 may extend through the implant body 210 from the posterior opening 214 to the anterior opening 216. The internal cavity 212 may be accessible via the posterior opening 214 and/or the anterior opening 216 and may be configured to receive bone graft material to promote fusion between the adjacent vertebrae.

The actuation mechanism 220 may include a drive feature 222 and a threaded portion 223, similar to the actuation mechanism 120 of the first embodiment. The first facet arm 230, second facet arm 231, and any additional wings may be configured to move within their respective facet arm channels 215, 217 between retracted and deployed configurations, allowing for minimally invasive insertion followed by lateral deployment for facet joint fixation.

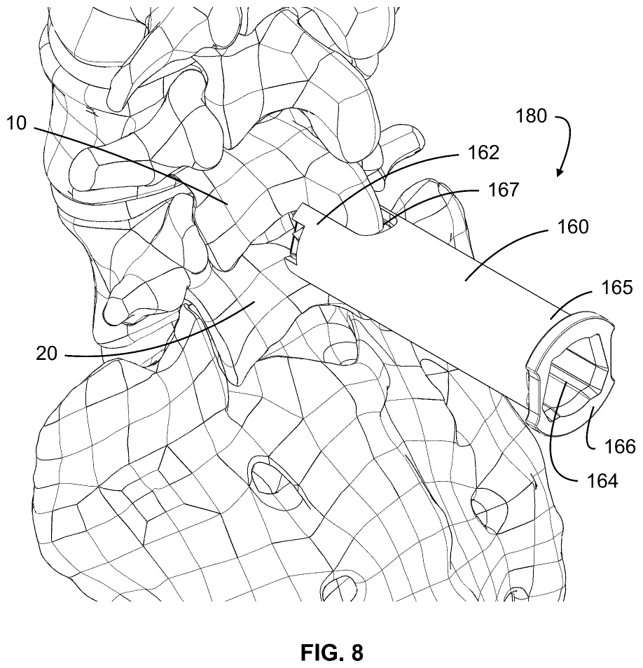

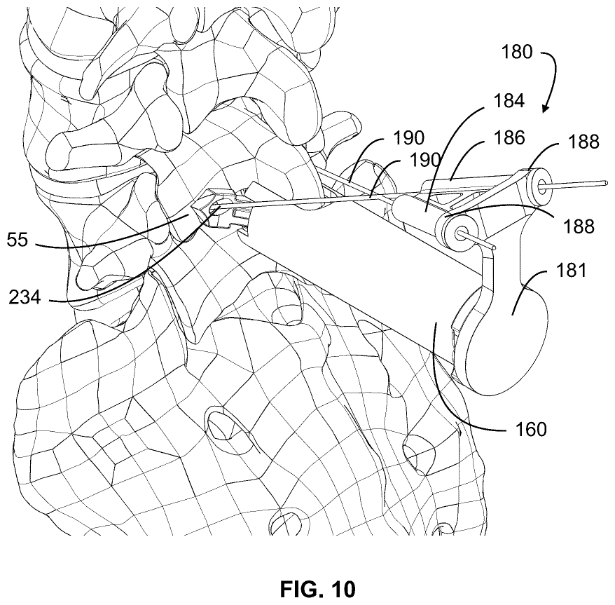

FIG. 8 is a perspective view of a guided access device 180 having a cannula 160, docked with an interspinous space between a superior vertebra and an inferior vertebra according to an embodiment of the present disclosure. The guided access device 180 may include the cannula 160 and a trajectory guide 181, as shown in FIG. 10. The cannula 160 may include a cannula distal end 162, a cannula channel 164, a cannula proximal end 165, a cannula attachment interface 166, and a cannula distal slot 167

The guided access device 180 may be provided for minimally invasive delivery and placement of the implant 100 or implant 200. The cannula 160 may be a rigid or semi-rigid hollow tube configured to be advanced through a small posterior incision to the interspinous space between a superior vertebra 10 and an inferior vertebra 20.

The cannula attachment interface 166 may be located at the cannula proximal end 165 and may comprise keyed slots, magnetic features, threaded connections, and/or other mechanical coupling features configured to secure additional instruments such as the trajectory guide 181 and/or to prevent unwanted rotation of the cannula 160 during use.

The cannula distal end 162 may be sized to allow passage of the implant in its retracted configuration along with associated components such as wings, arms, and the actuation mechanism. The cannula distal end 162 of the cannula 160 may be advanced to abut the spinous processes. The cannula distal end 162 may include the cannula distal slot 167 configured to receive the spinous processes and position the guided access device 180 relative to the spinous processes. The cannula 160 may create a cannula channel 164, through which a surgeon may introduce the interspinous implant, locking nut, wings, driver 170, and/or trajectory guide 181.

The cannula 160 may provide tissue retraction and create a protected working corridor through which surgical instruments can be introduced to manipulate the implant. The cannula 160 may also include anti-rotation features such as flats or teeth to prevent twisting during instrumentation.

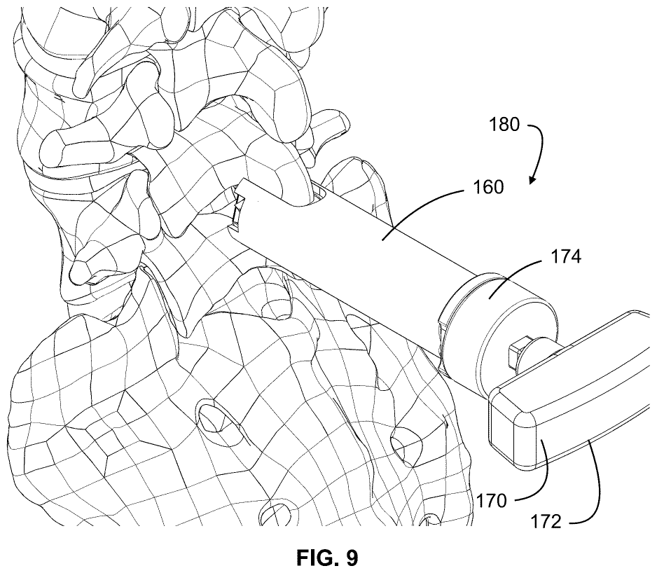

FIG. 9 is a perspective view of a driver 170 received in the guided access device 180 according to an embodiment of the present disclosure. The driver 170 may include a driver handle 172 and a driver torque-limiting mechanism 174.

As shown in FIG. 9, the driver 170 may be provided to engage and rotate the actuation mechanism 120 or 220. The driver handle 172 may be configured to be gripped by the surgeon to provide manual torque. A shaft may extend through the cannula 160 to reach the implant. An engagement feature at a distal end of the driver 170 may be configured to engage the drive feature 122 or 222 of the actuation mechanism, allowing rotation of the actuation mechanism to expand the implant, deploy the arms, and/or tighten the wing mechanism 149, 249.

In some embodiments, the driver 170 may include the driver torque-limiting mechanism 174 to prevent over-tightening of the actuation mechanism and potential damage to the implant or surrounding tissues. The driver 170 may be cannulated to allow insertion over a guidewire if desired for additional surgical guidance.

FIG. 10 is a perspective view of the guided access device 180 having a trajectory guide 181 and guidewire 190 according to an embodiment of the present disclosure. The trajectory guide 181 may include a guide attachment interface 182, a first guide channel 184, a second guide channel 186, and an angle defining feature 188.

The trajectory guide 181 may be provided to enable guided placement of bone fasteners, for example bone fastener 194. The guide attachment interface 182 may be configured to attach to the cannula attachment interface 166 or directly to the implant body. The first guide channel 184 and the second guide channel 186 may include tubular passages or bores through which drill bits, guidewires 190, and/or other instruments can be passed. The angle defining features 188 may establish the trajectory of each guide channel 184, 186 to align with target anatomical structures such as facet joints, laminae, pars interarticularis, or pedicles.

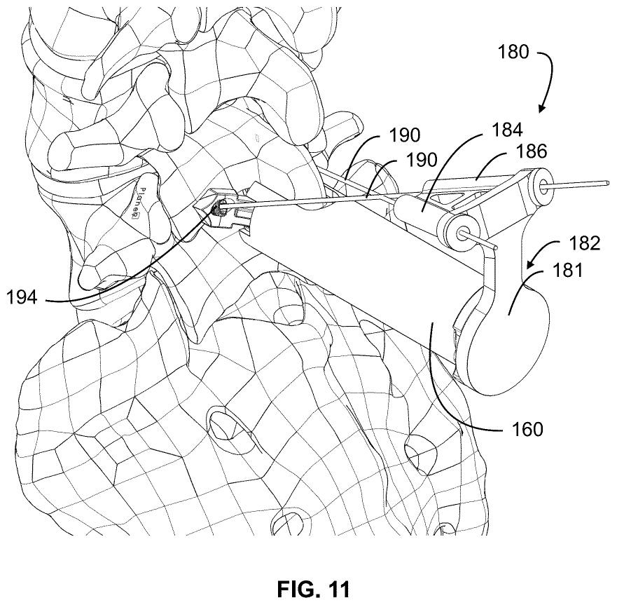

FIG. 11 is a perspective view of the guided access device 180 and bone fasteners 194 according to an embodiment of the present disclosure. One or more bone fasteners 194 may be placed through the trajectory guide 181. A guidewire 190 may first be advanced through a guide channel 184, 186 to the target location. A cannulated drill or reamer may then be advanced over the guidewire 190 to prepare a pilot hole.

Subsequently, a bone fastener 194, such as a screw, may be advanced over the guidewire 190 and into the prepared hole to secure the implant to the vertebra and/or to anchor the deployed facet arms to the facet joints. This guided approach enables percutaneous placement of supplemental fixation without requiring separate open surgical exposure.

By guiding the drill and fasteners, the system may enable percutaneous fastener placement into the facet, pars, translaminar, or pedicle without needing a separate open exposure. The guide may include color coding or indexing to ensure correct placement. The completed construct may thus have the interspinous implant clamped to the spinous processes, lamina arms locked under the lamina, and fasteners securing the implant to the vertebra on one or both sides.

The bone fasteners 194 may include self-tapping screws, self-drilling screws, cortical screws, cancellous screws, or other suitable fixation elements. The fasteners may be made from titanium alloys, stainless steel, or other biocompatible materials. In some embodiments, the fasteners may include radiopaque markers to facilitate visualization of their position during and after placement.

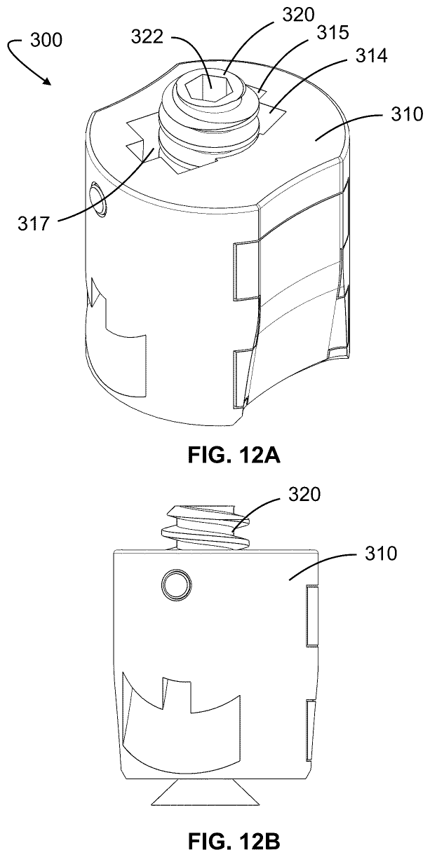

FIG. 12A is a perspective view of an implant body 310 of a minimally invasive spinal fusion implant 300 according to an embodiment of the present disclosure. FIG. 12B is a side view of the implant body 310. The various components of the implant 300 with the same reference numbers in the 300 series as those of the implant 100 in the 100 series may be similar in function and/or configuration to their counterparts of the implant 100. Differences between the implant 300 and the implant 100 will be set forth below.

The minimally invasive spinal fusion implant 300 may include the implant body 310, an actuation mechanism 320, an anchor block 326 having a wedge 327, and a retention clamp 328. The implant body 310 may include a posterior opening 314, a first facet arm channel 315, and a second facet arm channel 317. The actuation mechanism 320 may include a drive feature 322, a threaded portion 323, and a groove 324.

In an embodiment, the implant 300 may represent a non-expanding or fixed-height embodiment in which the implant body 310 maintains a substantially constant superior-inferior dimension during deployment. This configuration may be advantageous in cases where the desired interspinous spacing is already achieved by the anatomical positioning or where gradual distraction is not required.

The actuation mechanism 320 may include the drive feature 322 at its proximal end, similar to the actuation mechanisms of the previous embodiments. However, in this embodiment, rotation of the actuation mechanism 320 may primarily control locking of wings, rather than deployment of arms.

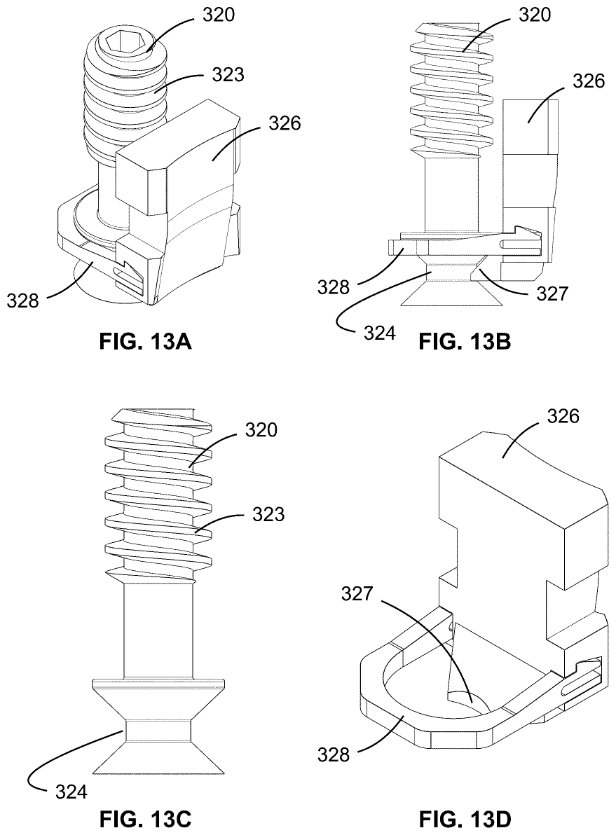

FIG. 13A is a perspective view of an actuation mechanism 320, an anchor block 326, and a retention clamp 328 of the implant body 310 according to an embodiment of the present disclosure. FIG. 13B is a side view of the actuation mechanism 320, the anchor block 326, and the retention clamp 328. FIG. 13C is a side view of the actuation mechanism 320. FIG. 13D is a perspective view of the anchor block 326 and the retention clamp 328.

The retention clamp 328 may be configured to be coupled with the anchor block 326 to capture the actuation mechanism 320. More specifically, the wedge 327 of the anchor block 326 may be positioned in the groove 324 of the actuation mechanism 320 so that the actuation mechanism 320 is captively coupled with the anchor block 326 and the retention clamp 328. The anchor block 326 and the retention clamp 328 may cooperate to limit translation of the actuation mechanism 320 along a longitudinal axis of the actuation mechanism 320.

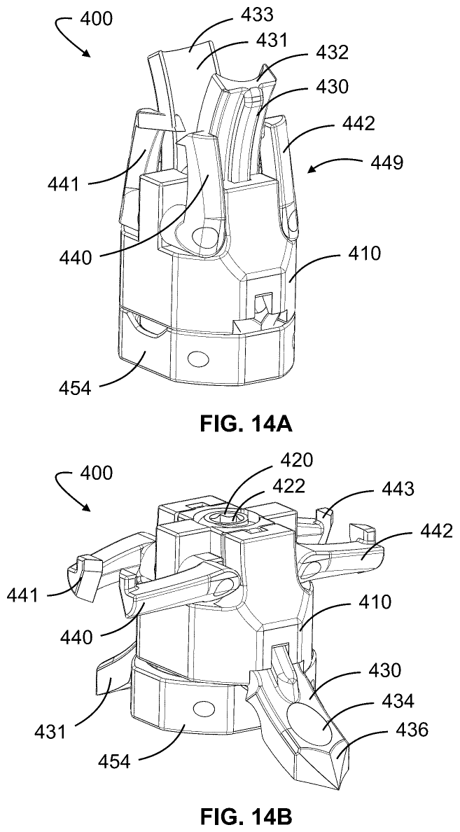

FIG. 14A is a perspective view of a minimally invasive spinal fusion implant 400 in a retracted configuration according to an embodiment of the present disclosure. FIG. 14B is a perspective view of the minimally invasive spinal fusion implant 400 in a deployed configuration according to an embodiment of the present disclosure. FIG. 15A is a top section view of the minimally invasive spinal fusion implant 400 in a deployed configuration. FIG. 15B is a front section view of the minimally invasive spinal fusion implant 400 in a deployed configuration.

The various components of the implant 400 with the same reference numbers in the 400 series as those of the implant 100 in the 100 series may be similar in function and/or configuration to their counterparts of the implant 100. Differences between the implant 400 and the implant 100 will be set forth below.

The minimally invasive spinal fusion implant 400 may include an implant body 410, an actuation mechanism 420, a first facet arm 430, a second facet arm 431, a first wing 440, a second wing 442, a third wing 441, and a fourth wing 443. The implant body 410 may include a first distraction portion 454. The actuation mechanism 420 may include a drive feature 422 and a threaded portion 423. The first wing 440, the second wing 442, the third wing 441, and the fourth wing 443 may cooperate to function as a wing mechanism 449 configured to engage the superior spinous process and the inferior spinous process.

The first facet arm 430 may include a first facet arm proximal portion 432, a first facet fixation feature 434, and a first facet arm distal portion 436. The second facet arm 431 may include a second facet arm proximal portion 433, a second facet fixation feature 435, and a second facet arm distal portion 437.

FIG. 14A shows the implant 400 in a retracted configuration suitable for minimally invasive insertion through a cannula. In this configuration, the first facet arm 430 and second facet arm 431 may be positioned substantially within or alongside the implant body 410, presenting a low profile that allows passage through the cannula without obstruction.

FIG. 14B shows the implant 400 in a deployed configuration in which the first facet arm 430 and second facet arm 431 extend laterally outward from the implant body 410. In the deployed configuration, the lateral extent of the implant 400 may be greater than the diameter of the delivery cannula to inhibit withdrawal of the implant through the cannula and providing mechanical resistance to migration.

Each of the first wing 440, the second wing 442, the third wing 441, and the fourth wing 443 may be pivotally coupled to the implant body 410, allowing each wing to rotate between retracted and deployed positions about a pivot axis.

The actuation mechanism 420 may interact with a cam follower or other actuation element driven by the actuation mechanism 420 to deploy the first facet arm 430 and the second facet arm 431. As the actuation mechanism 420 rotates, the cam follower may ride along a cam surface, progressively forcing the first facet arm 430 and the second facet arm 431 to pivot outward from the implant body 410. The geometry of the cam surface may be configured to provide controlled, gradual deployment with appropriate mechanical advantage.

The first facet arm distal portion 436 may be configured to engage the inferior aspect of the lamina of the superior vertebra when the first facet arm 430 is in the deployed position. Similarly, the second facet arm distal portion 437 may be configured to engage the inferior aspect of the lamina of the superior vertebra when the second facet arm 431 is in the deployed position. This engagement may provide resistance to flexion and extension motions of the spine, as well as rotational stability.

The first facet fixation feature 434 may be configured to receive a bone fastener to secure the first facet arm 430 to a facet joint or other anatomical structure. The first facet fixation feature 434 may be positioned such that when the first facet arm 430 is in the deployed position, the first facet fixation feature 434 aligns with the first guide channel 184 for percutaneous screw placement.

The second facet arm 431 may be configured similarly to the first facet arm 430, potentially as a mirror image, to provide bilateral engagement with the laminae or facet joints. The implant body 410 may include corresponding features to support both wings and allow their coordinated deployment through a common actuation mechanism.

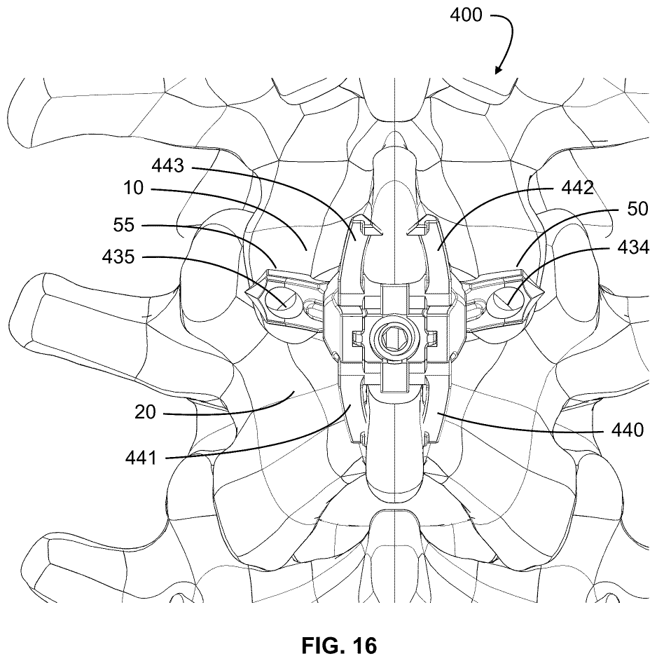

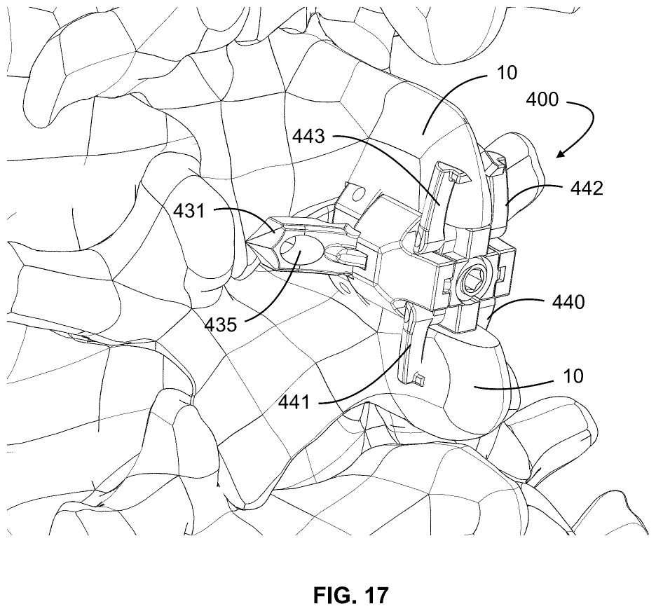

FIG. 16 is a perspective view of the minimally invasive spinal fusion implant 400 deployed in an interspinous space between a superior vertebra 10 and an inferior vertebra 20 according to an embodiment of the present disclosure. FIG. 17 is a perspective view of the minimally invasive spinal fusion implant 400 deployed in an interspinous space between a superior vertebra 10 and an inferior vertebra 20 according to an embodiment of the present disclosure.

As shown in FIGS. 16 and 17, the implant 400 may be deployed in an interspinous space between a superior vertebra 10 and an inferior vertebra 20. FIG. 16 illustrates the implant 400 with the facet arms 430, 431 deployed under the lamina of the superior vertebra 10, and with the wings 440, 441,442, 443 engaging the adjacent spinous processes.

FIG. 17 provides an alternative perspective view of the implant 400 deployed between the superior vertebra 10 and inferior vertebra 20, showing how the deployed facet arms 430, 431 extend laterally to engage the vertebral anatomy and provide rotational stability. The combination of interspinous clamping via the wings 440, 441, 442, 443 and laminar engagement via the facet arms 430, 431 may provide multi-axis stabilization of the spinal segment, resisting flexion, extension, lateral bending, and axial rotation.

Various modifications may be contemplated without departing from the invention. In one alternative (FIG. 12A-12B), the implant body may be a fixed height (non-distracting). The arms and wings may still be used to lock the spine. The lamina-engaging wings could be driven by rack-and-pinion, ratchet, cable, or spring mechanisms. They may slide and/or pivot relative to the implant body.

More than two wings (e.g., anterior and lateral) or one wing on a single side may be used. Wings could also be configured to engage the superior lamina of the inferior vertebra. The wings may be hinged or curved differently. For example, one wing could pivot down from above, clamping both spinous processes between it and the base. Plate-style wings could be integrated.

Wing 440, 441, 442, 443 surfaces may be toothed, smooth, or hybrid. The implant body may have variable porosity regions. Two or more implants can be connected by telescoping link bars to stabilize multiple levels. The trajectory guide 181 may include adjustable-angle sleeves or multiple keyed positions to allow different screw trajectories. Combinations of metal (titanium), polymer (PEEK), and bioresorbable materials may be used in different parts.

The invention may not be limited to the exact embodiments shown. For instance, a non-threaded (ratchet) version of the actuation mechanism could be used in place of a screw. The wings could snap onto the implant body or slide in a dovetail. The number and arrangement of arms and wings may vary. All such variations may be intended to fall within the scope of the claims.

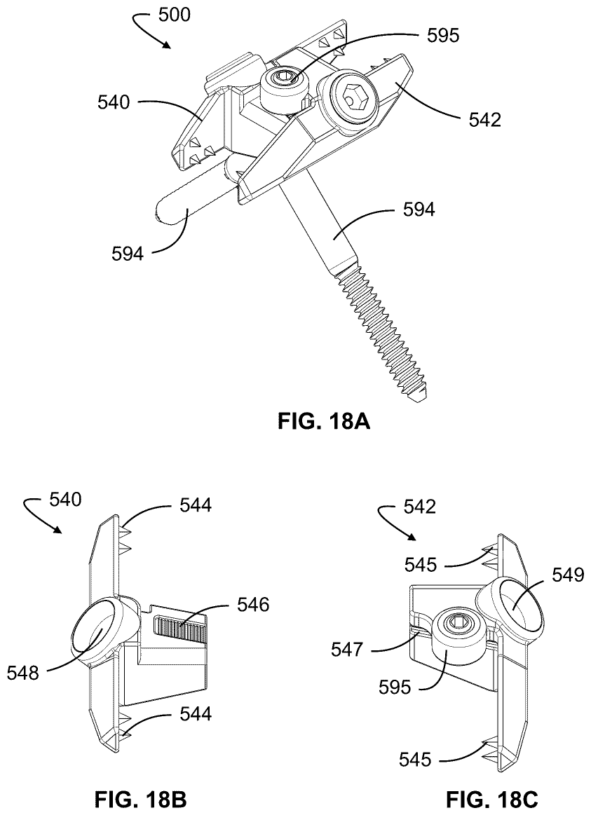

FIG. 18A is a perspective view of a minimally invasive spinal fusion implant 500 and bone fasteners 594 according to an embodiment of the present disclosure. FIG. 18B is a perspective view of a first clamp member 540 of the minimally invasive spinal fusion implant 500 according to an embodiment of the present disclosure. FIG. 18C is a perspective view of a second clamp member 542 of the minimally invasive spinal fusion implant 500 according to an embodiment of the present disclosure.

The minimally invasive spinal fusion implant 500 according to a fifth embodiment represents a variation of the previous embodiments. Unlike the implants 100, 200, 300, and 400, the implant 500 may not include an implant body or an actuation mechanism. Instead, the implant 500 may include a first clamp member 540, a second clamp member 542, and a clamp fastener 595 that may couple the two clamp members together. The implant 500 may be specifically designed for use at the L5-S1 joint, where the unique anatomical features of the lumbosacral junction require a different fixation approach.

The first clamp member 540 may include a first clamp bone engaging feature 544, a first clamp engagement portion 546, and a first clamp aperture 548. The first clamp bone engaging feature 544 may be configured to engage the spinous process of the L5 vertebra or the sacral midline crest. The first clamp bone engaging feature 544 may include spikes, serrations, ridges, or other surface textures to enhance purchase on the bone and resist migration.

The first clamp engagement portion 546 may be configured to mate with the second clamp member 542. The engagement portion 546 may provide a mechanical interface that allows the clamp fastener 595 to draw the two clamp members together and maintain compression on the spinous processes. The engagement portion 546 may include surfaces or features that ensure proper alignment of the first and second clamp members during tightening.

The first clamp aperture 548 may be configured to receive the bone fastener 594. The aperture 548 may extend through the first clamp member 540 to allow the bone fastener 594 to pass through and engage with a bone.

The clamp fastener 595 may comprise a threaded screw, bolt, or other mechanical coupling element configured to join the first clamp member 540 to the second clamp member 542. Tightening the clamp fastener 595 may draw the two clamp members toward each other, compressing the spinous processes between them. The fastener may include a head with a tool interface such as a hex socket or other drive feature to facilitate installation and adjustment.

The clamp fastener 595 may be received in an aperture that may be sized and positioned to facilitate proper alignment of the clamp members relative to the spinous processes during installation.

As shown in FIG. 18C, the second clamp member 542 may include a second clamp bone engaging feature 545, a second clamp engagement portion 547, and a second clamp aperture 549. The second clamp member 542 may be substantially similar to the first clamp member 540 but configured to engage the opposing side of the spinous processes. The second clamp bone engaging feature 545 may likewise include serrations, ridges, or textures to grip the bone surface and prevent slippage.

The second clamp engagement portion 547 may be configured to mate with the first clamp engagement portion 546 to form a secure coupling between the two clamp members. When the clamp fastener 595 is tightened through an aperture, the engagement portions 546, 547 may cooperate to maintain alignment and distribute clamping forces evenly across the spinous processes. The aperture may be threaded to receive the clamp fastener 595, or the fastener may be secured with a nut or other retention mechanism.

The implant 500 may be specifically designed for the L5-S1 joint, where the transition from the lumbar spine to the sacrum creates unique anatomical considerations. The L5 spinous process and sacral midline crest may vary significantly in morphology compared to typical lumbar spinous processes. The simplified two-piece clamp design of the implant 500 may be particularly suited to accommodate this anatomical variation while providing adequate stabilization.

The absence of an implant body and actuation mechanism in the implant 500 may provide several advantages for the L5-S1 application. The simplified design may reduce the overall profile of the implant, potentially minimizing soft tissue irritation. The direct coupling of the clamp members via the clamp fastener 595 may also allow for more direct load transfer between the spinous processes, which may be beneficial given the biomechanical demands at the lumbosacral junction. Additionally, the streamlined design may facilitate surgical placement in the anatomically constrained space of the lumbosacral region.

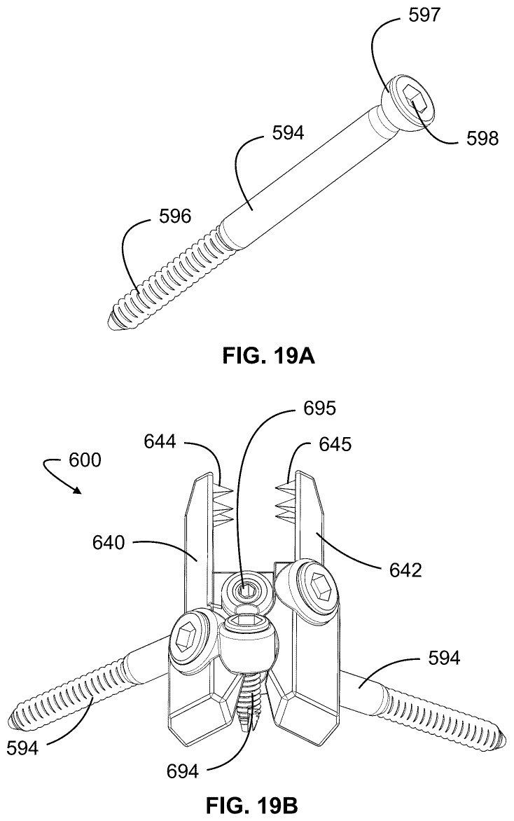

FIG. 19A is a perspective view of the bone fastener 594. The bone fastener 594 may include a threaded portion 596, a head portion 597, and a drive portion 598. FIG. 19B is a perspective view of a minimally invasive spinal fusion implant 600 and bone fasteners 594 according to an embodiment of the present disclosure. The minimally invasive spinal fusion implant 600 may include a first clamp member 640 having a plurality of first clamp bone engaging feature 644, a second clamp member 642 having a plurality of second clamp bone engaging features 645, and a clamp fastener 695.

With reference to FIGS. 19A and 19B, a minimally invasive spinal fusion implant 600 according to a sixth embodiment may be similar to the implant 500 but with additional fixation features. Like the implant 500, the implant 600 does not include an implant body or actuation mechanism. The implant 600 shares the simplified two-piece clamp design of the implant 500 but incorporates supplemental bone screw fixation to enhance stability, particularly at the L5-S1 joint.

The first clamp member 640 may include a first clamp engagement portion, and a first clamp aperture configured to receive the clamp fastener 695. The first clamp bone engaging feature 644 may be configured to engage the L5 spinous process and may include spikes, serrations, ridges, or surface textures to enhance grip on the bone and resist migration.

The second clamp member 642 may include a second clamp engagement portion, and a second clamp aperture. The second clamp member 642 may be substantially similar to the first clamp member 640 but configured to engage the opposing side of the spinous processes. When the clamp fastener 695 is tightened through the apertures in both clamp members, the engagement portions cooperate to compress the spinous processes and maintain alignment.

As shown in FIGS. 19A and 19B, the implant 600 may include bone fasteners 694 configured to provide supplemental fixation to the sacrum. The bone fasteners 594, 694 may extend through one or both of the clamp members to anchor directly into the sacral bone. This supplemental fixation may be particularly advantageous at the L5-S1 joint, where the sacral anatomy differs from typical lumbar vertebrae and may benefit from direct screw purchase into the dense sacral bone.

The bone fasteners 594, 694 may include screw heads that seat against the clamp members when fully inserted. The screw heads may have a drive portion 598 such as a hex socket, Torx recess, or other drive feature to allow insertion and tightening with standard surgical instruments. The bone fasteners 594, 694 may comprise self-tapping screws, self-drilling screws, or other suitable fasteners with thread profiles optimized for engagement with sacral bone.

The bone fasteners 594, 694 may comprise threads, cutting flutes, or other engagement elements configured to securely anchor the fasteners in bone. The thread profile may be designed to maximize contact with both the cortical and cancellous bone of the sacrum. In some embodiments, the screws may have a cannulated design to allow insertion over a guidewire or to provide a pathway for bone cement injection in cases of compromised bone quality.

The threaded portion 596 may extend from the head portion 597 and may be of sufficient length to achieve adequate purchase in the sacral bone. The threaded portion 596 may extend along a substantial portion of the shaft length to provide multiple threads of engagement. The thread pitch and diameter may be selected to optimize holding strength in the sacral bone structure.

Fastener tips may be configured as self-drilling or self-tapping tips to facilitate insertion without requiring pre-drilling in some cases. Alternatively, the tips may be designed for insertion into pre-drilled pilot holes to allow more controlled placement and trajectory planning. The tip geometry may be optimized to penetrate the dense cortical bone of the sacrum while minimizing the risk of iatrogenic injury.

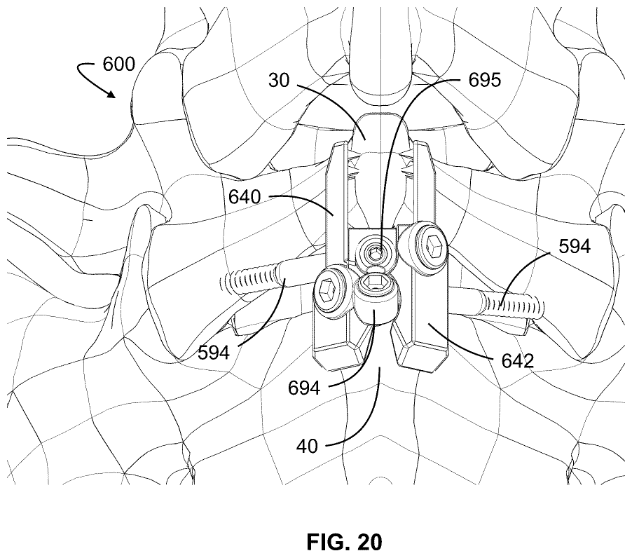

FIG. 20 is a perspective view of the minimally invasive spinal fusion implant 600 and bone fasteners 594 deployed in an interspinous space between a L5 vertebra 30 and a sacrum 40 according to an embodiment of the present disclosure.

As shown in FIG. 20, the implant 600 may be particularly well-suited for deployment at the lumbosacral junction between the L5 vertebra 30 and the sacrum 40. The L5-S1 level presents unique anatomical challenges due to the transitional morphology and the altered angulation at the lumbosacral junction. The sacrum 40 may have a variable midline crest with less prominent spinous tubercles compared to the lumbar spinous processes, making purely clamp-based fixation potentially insufficient.

The combination of clamp compression and direct bone screw fixation in the implant 600 may provide enhanced stability in this anatomically challenging region. The clamp members 640, 642 may engage both the L5 spinous process and the sacral midline structures, while the bone fasteners 594, 694 may be angled to engage the dense cortical bone of the sacrum, the sacral ala, or the lateral masses, which provide superior bone stock compared to the midline crest.

The dual fixation mechanism of the implant 600 may resist both translational and rotational motion at the L5-S1 junction. The clamp members provide bilateral compression to stabilize the spinous processes, while the bone fasteners 594, 694 provide direct anchoring to the sacrum that resists pull-out forces and toggling. This hybrid fixation approach may be especially beneficial in patients with osteoporosis, degenerative changes, or other conditions that compromise bone quality at the lumbosacral junction.

While the implant 600 is particularly suited for the L5-S1 level, it may also be used at other spinal levels where supplemental bone screw fixation is desired. The addition of the bone fasteners 594, 694 to the basic two-piece clamp design of the implant 500 provides versatility to address varying patient anatomies and bone quality conditions.

Several methods of use may be encompassed by the invention. In a typical procedure, a small posterior incision may be made and the guided access device 180 may be introduced to the target level under fluoroscopy. The assembled implant body 110 (with arms retracted and wings held open) may be inserted through the cannula into the interspinous space.

The actuation mechanism 120 may be turned via the driver 170 to lift the implant body, thereby distracting the interspinous space and restoring disc height. This may also typically tension the facet arm linkage. The facet arms may be deployed by further rotation of the shaft. The facet arms may extend laterally under the lamina of the vertebra above, engaging it securely (see FIGS. 14A-14B, 15A-15B, 16).

With the facet arms in place, the first and second wings may be slid into position on either side of the spinous processes. The locking component may be then threaded onto the shaft and tightened. This may draw the wings together, compressing the spinous processes between them and locking the implant. The wing bone engaging features may bite into the spinous processes, preventing migration.

Bone graft material (autograft, demineralized bone matrix, etc.) may be packed into the internal cavity either before insertion or after the implant may be in place. The trajectory guide may be attached to the implant or cannula. Drills or guidewires may be passed through the guide channels into the facet joints or lamina according to the pre-determined trajectories (FIGS. 10-11). Bone fasteners may then be inserted along these paths to anchor the implant to the vertebrae.

Alternatively, the guide may attach to the cannula and guide a trajectory lateral to the spine. The driver and guide may be removed, and the incision may be closed. The implant may maintain interspinous distraction, laminar fixation, and fused bone in the chamber.

This method may provide a hybrid stabilization: compression of the interspinous space (for decompression), laminar anchoring for rotational stability, and screw fixation for rigidity, all through one minimally invasive approach.

Those of skill in the art will recognize that this is only one of many potential methods that may be used to deploy a spinal fusion implant using minimally invasive techniques. In alternative embodiments, different methods may be used to deploy a spinal fusion implant using minimally invasive techniques other than the methods described above. Further, the methods set forth above may be used to secure other spinal fusion implants besides those specifically disclosed herein.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

The phrases “generally parallel” and “generally perpendicular” refer to structures that are within 30° parallelism or perpendicularity relative to each other, respectively. Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

What is claimed is:

1. A system for stabilizing a joint between a superior vertebra and an inferior vertebra, the system comprising:
an implant comprising:
a body configured to be implanted between a superior spinous process of the superior vertebra and an inferior spinous process of the inferior vertebra; and
a facet arm comprising a facet fixation feature, wherein the facet arm is movable between:
a retracted configuration in which the facet arm is housed within the body; and
a deployed configuration in which the facet arm extends laterally from the body to position the facet fixation feature in alignment with a facet joint to facilitate coupling of the facet arm to the facet joint.

2. The system of claim 1, wherein, in the retracted configuration, the implant is configured to pass through a cannula to engage the superior spinous process and the inferior spinous process.

3. The system of claim 2, wherein, in the deployed configuration, the implant is sized to inhibit passage through the cannula.

4. The system of claim 1, wherein the body comprises an internal cavity configured to receive bone graft material.

5. The system of claim 1, further comprising a fastener configured to be received in the facet joint;
wherein:
the facet fixation feature comprises an aperture configured to receive the fastener; and
in the deployed configuration, the fastener is insertable through the aperture and into the facet joint.

6. The system of claim 1, wherein the implant further comprises a wing mechanism configured to engage the superior spinous process and the inferior spinous process.

7. The system of claim 1, further comprising a guided access device configured to be docked with an interspinous space between the superior vertebra and the inferior vertebra;
wherein the guided access device comprises a cannula configured to receive the implant and direct the implant to the interspinous space.

8. The system of claim 7, wherein:
the facet fixation feature comprises an aperture configured to be aligned with the facet joint; and
the guided access device further comprises a trajectory guide comprising a longitudinal axis that is aligned with the aperture.

9. A system for stabilizing a joint between a superior vertebra and an inferior vertebra, the system comprising:
an implant comprising:
a body;
a wing mechanism configured to engage a superior spinous process and an inferior spinous process; and
a first actuation mechanism configured to engage the wing mechanism;
wherein:
actuation of the first actuation mechanism expands at least a portion of the body to distract an interspinous space between the superior vertebra and the inferior vertebra; and
further actuation of the first actuation mechanism deploys the wing mechanism to engage the superior spinous process and the inferior spinous process.

10. The system of claim 9, wherein the implant further comprises a facet arm comprising a facet fixation feature configured to facilitate coupling of the facet arm to a facet joint.

11. The system of claim 10, wherein the implant further comprises a second actuation mechanism configured to engage the facet arm, wherein actuation of the second actuation mechanism extends the facet arm laterally relative to the body to position the facet fixation feature in alignment with a facet joint.

12. The system of claim 10, wherein the facet arm is movable between:
a retracted configuration in which the facet arm is housed within the body; and
a deployed configuration in which the facet arm extends laterally from the body to position the facet fixation feature in alignment with a facet joint to facilitate coupling of the facet arm to the facet joint.

13. The system of claim 12, further comprising a fastener configured to be received in the facet joint;
wherein:
the facet fixation feature comprises an aperture configured to receive the fastener; and
in the deployed configuration, the fastener is insertable through the aperture and into the facet joint.

14. The system of claim 9, further comprising a guided access device configured to be docked with the interspinous space;
wherein the guided access device comprises a cannula configured to receive the implant and direct the implant to the interspinous space.

15. The system of claim 14, wherein:
the implant further comprises a facet arm comprising a facet fixation feature configured to facilitate coupling of the facet arm to a facet joint;
the facet fixation feature comprises an aperture configured to be aligned with the facet joint; and
the guided access device further comprises a trajectory guide comprising a longitudinal axis that is aligned with the aperture.

16. A system for stabilizing a joint between a superior vertebra and an inferior vertebra, the system comprising:
an implant comprising:
a body configured to be implanted between a superior spinous process of the superior vertebra and an inferior spinous process of the inferior vertebra;
a first facet arm;
a second facet arm; and
a wing mechanism configured to engage the superior spinous process and the inferior spinous process; and
a guided access device configured to be docked with an interspinous space between the superior vertebra and the inferior vertebra;
wherein:
the guided access device comprises a cannula configured to receive the implant and direct the implant to the interspinous space;
the implant is configured to pass through the cannula to engage the superior vertebra and the inferior vertebra;
the first facet arm comprises a first aperture configured to be aligned with a first facet joint;
the second facet arm comprises a second aperture configured to be aligned with a second facet joint;
the guided access device further comprises a first trajectory guide comprising a first longitudinal axis that is alignable with the first aperture; and
the guided access device further comprises a second trajectory guide comprising a second longitudinal axis that is alignable with the second aperture.

17. The system of claim 16, further comprising:
a first fastener configured to be received in the first facet joint; and
a second fastener configured to be received in the second facet joint;
wherein:
the first fastener is insertable through the first aperture and into the first facet joint; and
the second fastener is insertable through the second aperture and into the second facet joint.

18. The system of claim 16, wherein the implant further comprises a first actuation mechanism configured to engage the wing mechanism;
wherein:
actuation of the first actuation mechanism expands at least a portion of the body to distract the interspinous space between the superior vertebra and the inferior vertebra; and
further actuation of the first actuation mechanism deploys the wing mechanism to engage the superior spinous process and the inferior spinous process.

19. The system of claim 16, wherein the implant further comprises a second actuation mechanism configured to engage the first facet arm and the second facet arm;
wherein:
actuation of the second actuation mechanism extends the first facet arm laterally in a first direction relative to the body to position the first aperture in alignment with the first facet joint; and
actuation of the second actuation mechanism extends the second facet arm laterally in a second direction opposite the first direction relative to the body to position the second aperture in alignment with the second facet joint.

20. The system of claim 16, wherein:
the first facet arm is movable between:
a retracted configuration in which the first facet arm is housed within the body; and
a deployed configuration in which the first facet arm extends laterally in a first direction from the body to position the first aperture in alignment with the first facet joint to facilitate coupling of the first facet arm to the first facet joint; and
the second facet arm is movable between:
the retracted configuration in which the second facet arm is housed within the body; and
the deployed configuration in which the second facet arm extends laterally in a second direction opposite the first direction from the body to position the second aperture in alignment with the second facet joint to facilitate coupling of the second facet arm to the second facet joint.
